(19) United States
(12) Patent Application Publication
(10) Pub. No.: US 2012/0270860 A1
(43) Pub. Date: Oct. 25, 2012

(54) METHODS FOR TREATING OR PREVENTING ALCOHOL-RELATED DISORDERS OR CRAVING-RELATED DISORDERS

(76) Inventors: Gihyun Yoon, Eden Prairie, MN (US); Suck Won Kim, Edina, MN (US)

(21) Appl. No.: 13/089,661

(22) Filed: Apr. 19, 2011

(51) Int. Cl. A61K 31/197; A61K 31/551; A61P 25/00; A61P 25/22; A61P 25/32; A61P 25/18

(52) U.S. Cl. 514/221; 514/562

(57) ABSTRACT

The invention provides methods for treating or preventing alcohol-related disorders or craving-related disorders comprising administering an effective amount of N-acetylcysteine or a pharmaceutically acceptable salt thereof to a patient to treat or prevent the alcohol or craving disorders. Methods for enhancing drug delivery and improving treatment outcomes are also described.

### FIELD OF THE INVENTION

This invention provides methods for treating and preventing alcohol-related or craving-related disorders by administering N-acetylcysteine to a patient.

### BACKGROUND OF THE INVENTION

Despite enormous morbidity and mortality associated with alcohol-related disorders, the effectiveness of current pharmacotherapy remains limited. Current medications approved for alcohol dependence have shown only modest benefits. For example, disulfiram is not widely prescribed due to poor compliance and its lack of efficacy, and both acamprosate and naltrexone has failed to demonstrate efficacy in several clinical trials. There is a need for better methods for the treatment of one or more alcohol-related disorders such as, for example, alcohol dependence, alcohol abuse, alcohol craving, alcohol withdrawal, alcohol-induced liver cell injury, alcohol-induced brain cell injury, or alcohol use relapse.

### SUMMARY OF THE INVENTION

In some embodiments, the present invention provides a method for treating alcohol-related disorders in a patient comprising administering an effective amount of N-acetylcysteine or a pharmaceutically acceptable salt thereof to the patient to treat one or more disorders, wherein the disorders comprise alcohol dependence, alcohol abuse, alcohol intoxication, alcohol withdrawal, alcohol-induced delirium, alcohol-induced persisting dementia, alcohol-induced persisting amnestic disorder, alcohol-induced psychotic disorder, alcohol-induced mood disorder, alcohol-induced anxiety disorder, alcohol-induced sexual dysfunction or alcohol-induced sleep disorder.

In other embodiments, the invention provides a method for preventing alcohol-related disorders in a patient comprising administering an effective amount of N-acetylcysteine or a pharmaceutically acceptable salt thereof to the patient to prevent one or more disorders, wherein the disorders comprise alcohol dependence, alcohol abuse, alcohol intoxication, alcohol withdrawal, alcohol-induced delirium, alcohol-induced persisting dementia, alcohol-induced persisting amnestic disorder, alcohol-induced psychotic disorder, alcohol-induced mood disorder, alcohol-induced anxiety disorder, alcohol-induced sexual dysfunction or alcohol-induced sleep disorder.

In still other embodiments, the invention provides a method for reducing alcohol use in a patient comprising administering an effective amount of N-acetylcysteine or a pharmaceutically acceptable salt thereof to the patient to reduce the patient's alcohol use.

In still other embodiments the invention provides a method of treating a hospitalized patient diagnosed with alcohol-related disorders or alcohol-related cell injury comprising administering 600 mg-8000 mg/day to the patient in an emergency department, detoxification unit or inpatient setting. In certain embodiments, N-acetylcysteine is administered parenterally to a patient in an intensive care unit alone or together with benzodiazepines, thiamine, and folic acid for treating both alcohol-related disorder and alcohol-related cell injury.

In yet other embodiments, the invention also provides a method for treating or preventing craving-related disorders in a patient comprising administering an effective amount of N-acetylcysteine to the patient to treat or prevent one or more craving disorders, wherein the craving disorders comprise food cravings and compulsive overeating, sexual cravings and hypersexual behaviors, or shopping cravings and compulsive shopping.

### DETAILED DESCRIPTION OF THE INVENTION

In some embodiments, the present invention provides methods for treating a patient with alcohol-related disorders by administration of a therapeutically effective amount of N-acetylcysteine. In an effort to develop an effective pharmacotherapy, N-acetylcysteine was used in a clinical study for the treatment of alcohol-related disorders. The clinical study focused on alcohol craving, which is considered one of the core components of alcohol-related disorders. In addition to alcohol craving, N-acetylcysteine was studied for the treatment of other craving-related disorders such as, for example, food cravings, sexual cravings, or shopping cravings.

N-acetylcysteine is an amino acid, antioxidant and procysteine drug, which efficiently support glutathione biosynthesis. N-acetylcysteine can be purchased without a prescription in health food stores, where it is typically promoted as improving mental functions. N-acetylcysteine may protect against hepatic^{1, 2} and brain cell injury.³ As a hepatoprotective agent, N-acetylcysteine has been used to prevent hepatic injuries caused by sulfur mustard,⁴ azathioprine (immunosuppressant drug),^{5, 6} endotoxin (lipopolysaccharide, LPS),⁷ 3-Butene-1,2-diol (BDD),⁸ and acetaminophen.⁹

Recently, N-acetylcysteine has been evaluated for the treatment of addictive disorders.^{10, 11} Several preclinical studies have demonstrated that N-acetylcysteine inhibited heroin-induced reinstatement¹² and cocaine-primed reinstatement^{13, 14} by stimulating cysteine/glutamate exchange. N-acetylcysteine significantly reduced cocaine use in an open-label clinical study of cocaine-dependent patients. ¹⁵ N-acetylcysteine was safe and well tolerated in this population. Similarly, in a double-blind placebo-controlled crossover trial, N-acetylcysteine reduced cocaine use and craving in cocaine dependent individuals.^{16, 17} N-acetylcysteine also showed the efficacy for the treatment of pathological gambling.¹⁸

In several animal studies, N-acetylcysteine may reduce alcohol use and toxicity.^{1, 3, 19, 20} However, there is no information available on the use of N-acetylcysteine to treat human subjects and there is no data available as to whether N-acetylcysteine is effective in treating alcohol craving or improving quality of life

In this application certain terms set out below have the following meanings or definitions.

“Alcohol-related disorders” include alcohol use disorders, alcohol-induced disorders and alcohol craving. Alcohol use disorders include but are not limited to alcohol dependence and alcohol abuse. Alcohol-induced disorders include but are not limited to alcohol intoxication, alcohol withdrawal, alcohol-induced delirium, alcohol-induced persisting dementia, alcohol-induced persisting amnestic disorder, alcohol-induced psychotic disorder, alcohol-induced mood disorder, alcohol-induced anxiety disorder, alcohol-induced sexual dysfunction, alcohol-induced sleep disorder and alcohol use relapse.

“Craving-related disorders” include (1) food cravings and compulsive overeating, (2) sexual cravings and hypersexual behaviors, and (3) shopping cravings and compulsive shopping.

“Alcohol craving” includes but is not limited to a physiological-based and/or psychological-based desire for alcohol, for example, alcoholic beverages.

“Food craving” includes but is not limited to a physiological-based and/or psychological-based desire for food.

“Sexual craving” includes but is not limited to a physiological-based and/or psychological-based desire for sex.

“Shopping craving” includes but is not limited to a physiological-based and/or psychological-based desire for shopping.

“Improves hepatic function”, includes but is not limited to decreased levels of a liver enzyme (e.g. aspartate aminotransferase, alanine aminotransferase or γ-glutamyl transferase). Levels of liver enzymes can be measured by liver function tests (e.g. aspartate aminotransferase liver function test, alanine aminotransferase liver function test or y-glutamyl transferase liver function test.

“Effective amount” or “therapeutically effective amount” means a sufficient amount of the compound to provide the desired therapeutic or prophylactic effect to a patient or individual. In the context of treating alcohol-related disorders or craving-related disorders, “effective amount” or “therapeutically effective amount” means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered. Those of skill in the art given the benefit of the present disclosure can easily determine appropriate effective amounts by taking into account metabolism, bioavailability, and other factors that affect plasma levels of N-acetylcysteine or a metabolite thereof following administration within the unit dose ranges disclosed further herein for different routes of administration.

“Treatment” or “treating” refers to any manner in which the symptoms of a condition, disorder or disease are ameliorated or otherwise beneficially altered. There are a variety of methods for diagnosing alcohol-related disorders. Other conventional diagnostic methods can be used as well. In the context of treating alcohol-related disorders or craving-related disorders, the disorder can be onset or relapsed. Full eradication of the disorder or symptoms or aspects of the disorder is not required. Amelioration of symptoms or aspects of a particular disorder refers to any lessening of symptoms or aspects, whether permanent or temporary, that can be attributed to or associated with administration of N-acetylcysteine. Treatment also encompasses pharmaceutical use of the compositions in accordance with the methods disclosed herein. After alcohol-related disorders or craving-related disorders are initially treated with N-acetylcysteine, pateints may be encouraged to take N-acetylcysteine for several months to several years to control or prevent their cravings and to prevent relapse. Patients may take N-acetylcysteine daily or as needed for this purpose. N-acetylcysteine will be even more effective for alcoholic patients with alcohol-induced liver cell injury because it may work as a liver-protective agent.

Currently, several different forms of N-acetylcysteine are prescribed for treating different clinical indications: (1) oral and intravenous N-acetylcysteine (Acetadote) as an antidote for the treatment of acetaminophen toxicity and (2) inhaled N-acetylcysteine (Mucomyst) as a mucolytic agent for the treatment of respiratory disorders.

N-acetylcysteine may be formulated as pharmaceutical composition and administered to a patient in a variety of forms adapted to the chosen route of administration, i.e., orally, topically or parenterally, by intravenous, intramuscular or subcutaneous routes. The invention provides a therapeutic method comprising administrating N-acetylcysteine orally (tablet, capsule, effervescent tablet or solution), parenterally (injection or infusion), by inhalation or by a transdermal patch to a patient with alcohol-related disorders and/or alcohol-induced liver cell injury.

N-acetylcysteine may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, N-acetylcysteine may be combined with one or more excipients and used in the form of ingestible tablets, buccal tablets, troches, capsules, elixirs, suspensions, syrups, wafers, and the like. The amount of N-acetylcysteine in such therapeutically useful compositions is such that an effective dosage level will be obtained.

The tablets, troches, pills, capsules, and the like may also contain the following suitable binders, disintegrating agents, lubricants, and sweetening agents. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain N-acetylcysteine, sucrose or fructose as a sweetening agent, methyl and propylparabens as preservatives, a dye and flavoring such as cherry or orange flavor.

In addition N-acetylcysteine may be incorporated into extended or sustained-release preparations and devices. The invention also provides a therapeutic and prevention method comprising administration of an N-acetylcysteine extended-release formulation for the treatment of alcohol-related disorders, or craving-related disorders, and/or alcohol-induced liver cell injury. As N-acetylcysteine has a short half-life, such an extended-release form of N-acetylcysteine may enhance compliance and effectiveness by reducing the number of dosing events per day.

Examples of making extended or sustained release pharmaceutical compositions are reported in Chattaraj et al., U.S. Pub. No. US2009/0238873, Nemeroff et al., U.S. Pub. No. US2002/0160056, and Raimondi, U.S. Pat. No. 6,207,182.

N-Acetylcysteine may also be administered intravenously or intraperitoneally by infusion or injection. Solutions of N-acetylcysteine or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

N-Acetylcysteine may also be administered intramuscularly every 2-4 weeks for a patient to improve compliance and efficacy.

The pharmaceutical dosage forms suitable for injection or infusion can include sterile aqueous solutions or dispersions or sterile powders comprising N-acetylcysteine that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For topical administration N-acetylcysteine may be applied in pure form. However, it will generally be desirable to administer N-acetylcysteine to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver compounds to skin are known to the art; for example, see Jacquet et al., U.S. Pat. No. 4,608,392, Geria, U.S. Pat. No. 4,992,478, Smith et al., U.S. Pat. No. 4,559,157 and Wortzman, U.S. Pat. No. 4,820,508.

The amount of N-acetylcysteine or an active salt or derivative thereof, required for use in treatment will vary not only with the particular salt selected but also with the route of administration, the nature of the condition being treated and the age and condition of the patient and will be ultimately at the discretion of the attendant physician or clinician.

N-Acetylcysteine is readily administered orally. Effective amounts or total doses of N-acetylcysteine include, for example, amounts or doses of about 600 to 8000 mg, alternatively amounts or doses of about 1200 to 4800 mg, or still other amounts or doses of about 2400 to 3600 mg.

If N-acetylcysteine is used intravenously for alcohol-related disorders, the total doses are smaller than those used for the treatment of acetaminophen overdose, which is: 1) loading dose: 150 mg/kg in 200 mL of diluent administered over 60 minutes; 2) second dose: 50 mg/kg in 500 mL of diluent administered over 4 hours; and 3) third dose: 100 mg/kg in 1000 mL of diluent administered over 16 hours.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. Some patients need to take N-acetylcysteine frequently, up to 8 times a day, in order to maximize treatment outcomes for treating alcohol-related disorders or craving-related disorders.

N-Acetylcysteine may also be administered in combination with other therapeutic agents, for example, other agents that are useful for the treatment of alcohol-related disorders. Examples of such agents include naltrexone, disulfiram, acamprosate, nalmefene, topiramate, baclofen, memantine, ondansetron and rimonabant. Other agents are useful for the treatment of food cravings or compulsive overeating. Examples of such agents include orlistat, sibutramine, rimonabant, naltrexone, topiramate, bupropion, phendimetrazine, phentermine, diethylpropion, zonisamide, quinagolide and lorcaserin. Other agents are useful for the treatment of sexual cravings and hypersexual behaviors. Examples of such agents include naltrexone, disulfiram, acamprosate, nalmefene, topiramate, baclofen, memantine, ondansetron or rimonabant. Other agents are useful for the treatment of shopping cravings and compulsive shopping. Examples of such agents include naltrexone, disulfiram, acamprosate, nalmefene, topiramate, baclofen, memantine, ondansetron or rimonabant.

The invention also provides a kit comprising N-acetylcysteine, or a pharmaceutically acceptable salt thereof, at least one other therapeutic agent, packaging material, and instructions for administering N-acetylcysteine or the pharmaceutically acceptable salt thereof and the other therapeutic agent or agents to a patient to treat alcohol-related disorders.

The invention also provides a therapeutic method comprising administrating to a hospitalized patient with alcohol-related disorders and/or alcohol-induced liver cell injury a therapeutically effective amount of N-acetylcysteine orally or parenterally. Currently alcohol-dependent patients do not receive N-acetylcysteine during their hospitalization. However, this invention teaches that N-acetylcysteine should be prescribed to some hospitalized patients for treating alcohol-related disorders and/or alcohol-related liver cell injury. For example, alcoholic patients can be treated with N-acetylcysteine in the emergency department, detoxification unit or inpatient settings. N-acetylcysteine can be prescribed orally (dose: 600 mg-8000 mg/day) or parenterally (less dosage needed than treating acetaminophen overdose) for alcohol-related disorders. N-acetylcysteine may be administered parenterally to a patient in an intensive care unit (ICU). N-acetylcysteine could speed up their recovery from alcohol-related disorders by normalizing the glutamate system. N-acetylcysteine can be used alone or can be added to the standard medications, such as benzodiazepines, thiamine, and folic acid, used for treating alcohol-related disorders. Also, N-acetylcysteine would be even more effective for patients with alcohol-induced liver cell injury, which is commonly present in alcoholic patients. This new approach of prescribing N-acetylcysteine may shorten inpatient length of stay by improving alcohol-related disorders and/or alcohol-induced liver cell injury.

Experimental results supporting the methods of the invention are described in Examples 1-8. For example, as described in Example 4, several alcohol-dependent individuals without liver disease improved their liver functions while taking N-acetylcysteine. Thus, N-acetylcysteine may be prescribed to an alcohol-dependent individual, regardless of the lack of current liver disease, to prevent alcohol-related liver disease. Also for example, in Examples 6-8, several individuals were able to decrease their cravings and craving-related behaviors while taking N-acetylcysteine. Thus, N-acetylcysteine may be prescribed to an individual to treat those cravings and craving-related behaviors.

### EXAMPLE 1

### Safety of N-acetylcysteine on Patients with Alcohol-Related Disorders

Alcohol-dependent individuals were recruited for participation in a randomized, double-blind, placebo-controlled trial for treating alcohol dependence. To evaluate the safety and efficacy of N-acetylcysteine, 2 groups (N-acetylcysteine 3600 mg/day and placebo) were compared. The total study duration was 9 weeks which included a 1-week screening period and an 8-week randomized study drug treatment period. Subjects were randomized to one of two groups (Table 1) and were evaluated weekly.

Of 46 subjects, 44 subjects who took at least one study medication were included in analysis. Data analyses were conducted on an intention-to-treat sample using the linear mixed effects model introduced by Laird and Ware (Laird, N. M. & J. H. Ware. 1982. “Random-Effects Models for Longitudinal Data.” Biometrics 38:963-974). Outcome variables were measured over nine consecutive weeks. The covariates were Alcoholics Anonymous attendance and baseline alcohol craving. All the P-values reported are 2-tailed. Significance level was set at p<0.05.

N-acetylcysteine was well tolerated. There were no serious adverse effects. No subjects were discharged from the study due to side effects. No subjects reduced the scheduled doses of N-acetylcysteine or needed interventions due to side effects.

TABLE 1
Medication schedule

| Week | N-acetylcysteine Group | Placebo Group |
|---|---|---|
| 1 | N-acetylcysteine 900 mg in am | matched placebo in am |
| 2 | N-acetylcysteine 900 mg in am | matched placebo in am |
| | N-acetylcysteine 900 mg in pm | matched placebo in pm |
| 3 | N-acetylcysteine 1800 mg in am | matched placebo in am |
| | N-acetylcysteine 900 mg in pm | matched placebo in pm |
| 4-8 | N-acetylcysteine 1800 mg in am | matched placebo in am |
| | N-acetylcysteine 1800 mg in pm | matched placebo in pm |

### EXAMPLE 2

### Effect of N-acetylcysteine on Quality of Life

The effect of N-acetylcysteine on quality of life was measured weekly during the trial described in Example 1. The quality of life was measured by the Quality of Life Enjoyment and Satisfaction Questionnaire (Q-LES-Q).

The short form of the Q-LES-Q is a 16-item self-rated scale designed to assess quality of life, differences in the degree of enjoyment and satisfaction among groups of subjects, as well as changes over time in a single subject (Endicott J., et al., Quality of Life Enjoyment and Satisfaction Questionnaire: a new measure, Psychopharmacol. Bull., 1993, 29(2), 321-326). It is a 5-point scale, with higher scores indicating better health status. The Q-LES-Q SF has been used extensively in drug treatment trials in psychiatry and addictions and has shown good reliability and validity (Endicott J., et al., Pediatric Quality of Life Enjoyment and Satisfaction Questionnaire (PQ-LES-Q): reliability and validity. J. Am. Acad. Child Adolesc. Psychiatry, 2006, 45(4), 401-7). It assesses the following areas: physical health, mood, work, household activities, social relationships, family relationships, leisure time activities, ability to function in daily life, sexual drive, interest and/or performance, economic status, living/housing situation, ability to get around physically, ability to do work or hobbies, and overall sense of well being.

Compared to placebo, N-acetylcysteine improved the quality of life during the trial as measured by the Quality of Life Enjoyment and Satisfaction Questionnaire (p=0.017) (Table 2).

TABLE 2
Quality of Life Enjoyment and Satisfaction as measured Q-LES-Q

Q-LES-Q

| Week | N-acetylcysteine | Placebo |
|---|---|---|
| 1 | 50.4000 | 52.7362 |
| 2 | 51.6390 | 55.6261 |
| 3 | 53.8772 | 54.7242 |
| 4 | 54.1789 | 56.8921 |
| 5 | 57.7852 | 56.7533 |
| 6 | 57.5529 | 56.7967 |
| 7 | 58.1422 | 56.8035 |
| 8 | 57.8000 | 57.5294 |
| 9 | 58.7244 | 56.3961 |

In light of the improved quality of life described above, one embodiment of the invention provides a method for improving the quality of life in a patient with alcohol-related disorders comprising administering to the patient a therapeutically effective amount of N-acetylcysteine.

### EXAMPLE 3

### Effect of N-acetylcysteine on Alcohol Craving

During the trial described in Example 1, alcohol craving was measured weekly by the Penn Alcohol Craving Scale (PACS)(Flannery B. A., et al., Psychometric properties of the Penn Alcohol Craving Scale. Alcohol, Clin. Exp. Res. 1999, 23(8), 1289-95). The PACS is a 5-item self-rated scale designed to assess alcohol craving severity (frequency, intensity, duration, resistance, and overall craving) during the preceding 1 week. Each item has a score range of 0-6 (maximum total craving score=30). The PACS has demonstrated excellent reliability and good construct/discriminant/predictive validity.

Alcohol craving was also measured weekly by the Obsessive Compulsive Drinking Scale (OCDS) (Anton R. F., et al., The obsessive compulsive drinking scale: A new method of assessing outcome in alcoholism treatment studies. Arch. Gen. Psychiatry. 1996, 53(3), 225-231; Anton R. F., et al., The Obsessive Compulsive Drinking Scale: a self-rated instrument for the quantification of thoughts about alcohol and drinking behavior, Alcohol Clin. Exp. Res., 1995, 19(1), 92-99). The OCDS is a 14-item self-rated reliable scale designed to assess obsessive and compulsive aspects of alcoholism. Each item has a score range of 0-4 (maximum total score=56). Studies of factor analysis have reported that the OCDS loads to three or four factors (OCDS: P<0.05) (Roberts. J. S., et al., Factor structure and predictive validity of the Obsessive Compulsive Drinking Scale, Alcohol Clin. Exp. Res., 1999, 23(9), 1484-1491; Bohn, M. J., et al., Psychometric properties and validity of the obsessive-compulsive drinking scale, Alcohol Clin. Exp. Res., 1996, 20(5), 817-23; Kranzler, H. R., et al., Validity of the Obsessive Compulsive Drinking Scale (OCDS): does craving predict drinking behavior?, Alcohol Clin. Exp. Res. 1999, 23(1), 108-14).

Compared to placebo, N-acetylcysteine decreased alcohol craving during the trial as measured by the Penn Alcohol Craving Scale (p=0.041) and the Obsessive Compulsive Drinking Scale (p=0.042) (Tables 3 and Table 4).

TABLE 3
Alcohol craving as measured PACS

PACS

| Week | N-acetylcysteine | Placebo |
|---|---|---|
| 1 | 18.1429 | 17.9565 |
| 2 | 15.1500 | 15.4348 |
| 3 | 14.0000 | 15.5455 |
| 4 | 11.9474 | 12.8095 |
| 5 | 11.7778 | 13.9000 |
| 6 | 9.3529 | 12.6000 |
| 7 | 8.3333 | 12.4737 |
| 8 | 7.2667 | 11.6471 |
| 9 | 7.2000 | 11.4706 |

TABLE 4
Alcohol craving as measured OCDS

OCDS

| Week | N-acetylcysteine | Placebo |
|---|---|---|
| 1 | 29.0000 | 27.6087 |
| 2 | 24.9000 | 23.0000 |
| 3 | 21.6316 | 22.7273 |
| 4 | 20.3684 | 20.2857 |
| 5 | 18.5556 | 20.1000 |
| 6 | 16.0000 | 19.4500 |
| 7 | 13.5333 | 19.3158 |
| 8 | 13.4667 | 16.7647 |
| 9 | 13.8000 | 16.7647 |

In light of the decreased alcohol craving described above, one embodiment of the invention provides a method for decreasing alcohol craving in a patient with alcohol-related disorders comprising administering to the patient a therapeutically effective amount of N-acetylcysteine.

### EXAMPLE 4

### Effect of N-acetylcysteine on Liver Functions in Alcohol-Related Disorders

The effect of N-acetylcysteine on liver functions was measured during the trial described in Example 1. Liver functions were assessed by measuring changes in plasma aspartate aminotransferase (AST), alanine aminotransferase (ALT) and γ-glutamyl transferase (GGT) levels three times during the study (week 0, week 5 and week 9).

N-acetylcysteine improved liver functions during this 8-week trial as measured by improved AST liver function tests, improved ALT liver function tests and improved GGT liver function tests (Table 5).

TABLE 5
Liver function tests

| | Week | | |
|---|---|---|---|
| | 0 | 5 | 9 |
| AST | 40.89 | 30.28 | 26.13 |
| ALT | 36.67 | 33.06 | 29.13 |
| GGT | 193.00 | 139.06 | 92.93 |

### EXAMPLE 5

### Effect of N-acetylcysteine on Alcohol Consumption and Alcohol-Related Symptoms

N-acetylcysteine reduced alcohol consumption and other alcohol-related symptoms in numerous test subjects as exemplified in the cases below.

### Case 1:

A 61-year-old married, retired man with a 42-year history of alcohol dependence had been drinking about 0.75 liter of rum daily for the past 10 years. He reported that he drank alcohol “to get a buzz and to relax.” He was able to stop drinking alcohol after taking 900 mg of N-acetylcysteine, but he still experienced a moderate level of alcohol craving 2-3 times a week. At 2700 mg/day, he reported much less alcohol craving “even when watching a TV beer commercial.” He was able to stay sober on N-acetylcysteine, “I did not drink at the Super Bowl party.” His anxiety and alcohol withdrawal symptoms were also reduced on N-acetylcysteine. He reported a substantial reduction in his craving on 3600 mg of N-acetylcysteine. “It's now very easy to stay sober.”

### Case 2:

A 60-year-old man with a 40-year history of alcohol dependence had been drinking about 10-15 drinks of vodka and beer daily for the past 15 years. He stated that he drank alcohol “to escape from my job and stress.” Soon after he was started on N-acetylcysteine, he was able to abstain from alcohol. “It was not that difficult not to drink.” He reported that his wife and children were “very pleased” about his sobriety. “I will stay sober for my children and my pride.” His liver function tests markedly improved (GGT: 1420 to 327, AST: 41 to 20, ALT: 51 to 18) in two months while taking N-acetylcysteine.

### Case 3:

A 42-year-old self-employed man with 3 previous alcohol treatments presented with severe alcohol craving. He had been drinking about 20 drinks of brandy and beer 5-6 times a week in the evening after work. After he was placed on 900 mg of N-acetylcysteine, he reported more stable mood and less craving. The frequency and severity of alcohol use decreased gradually. At 3600 mg/day, he was able to stop drinking alcohol completely. “Maybe I am taking a miracle drug.” He reported a substantial improvement in his craving, sleep and his thought process. “Now I don't drink alcohol anymore when I see my friends.”

### Case 4:

A 44-year-old full-time employed man with a history of 9 previous DUIs had been drinking 12-24 beers/day by himself at a bar or home to control his stress and anxiety, and “to feel better.” However, he wanted to quit alcohol “not to disappoint my family and myself.” When 900 mg of N-acetylcysteine was started, he was able to reduce his alcohol use to 6 drinks/day. Several weeks later, he was able to abstain from alcohol while taking 2400-3600 mg of N-acetylcysteine. He was also satisfied as he could control his anxiety much better on N-acetylcysteine; “my anxiety is gone.”

### Case 5:

A 57-year-old businessman noticed that his alcohol use caused performance problems at work and relationship problems at home with his wife. He presented with a goal of cutting down his vodka consumption from 6-12 drinks to less than 3 drinks per day. At 900-2700 mg/day, his drinking consumption was about the same. However, he reduced his drinking significantly after N-acetylcysteine was raised to 3600 mg. He reported that he was able to stop after having 2-3 shots of vodka. “I don't trust my willpower. I know that I am able to do it because of N-acetylcysteine.”

In light of the decreased alcohol use as described above, one embodiment of the invention provides a method for decreasing alcohol use in a patient comprising administering to the patient an effective amount of N-acetylcysteine or a pharmaceutically acceptable salt thereof.

### EXAMPLE 6

### Effect of N-acetylcysteine on Food Cravings and Compulsive Overeating

N-acetylcysteine reduced food cravings and compulsive overeating in numerous test subjects as exemplified in the cases below.

### Case 1:

A 57-year-old self-employed man with diabetes, hypertension, hypercholesterolemia, alcohol dependence, and obesity (239 lbs, BMI: 35.3) participated in the study of N-acetylcysteine for treating alcohol dependence. During the study, he noticed that his food craving was better controlled while taking 3600 mg of N-acetylcysteine. He had tried to lose his weight, but his weight remained about 240 lbs for some time. He used to eat a large portion of food several times a day, especially while working in his home office. Since he was placed on 2700-3600 mg of N-acetylcysteine per day, he was able to restrict his food and alcohol intake much better. His weight decreased from 239 lbs to 225 lbs in 5 weeks, and he was able to maintain at 224 lbs two months later while taking 3600 mg of N-acetylcysteine. He was “satisfied” with this outcome.

### Case 2:

A 42-year-old man with obesity (295 lbs, BMI: 41.1) was started on N-acetylcysteine. After taking 900-2700 mg of N-acetylcysteine per day, he lost only a few pounds (his weight ranged from 291 lbs to 294). When N-acetylcysteine was increased to 3600 mg/day, he reported much less food craving. He stated that he was able to control his food intake much better and felt “more confident” mentally and physically. His weight went down from 295 lbs to 286 lbs in one month while taking N-acetylcysteine. During this period, he did not initiate any exercise. He planned to start a weight loss program and keep taking N-acetylcysteine.

Case 3:
A 26-year-old woman began to struggle with excessive food intake (usually various carbohydrates) from age 13. Prior to taking N-acetylcysteine, her weight was 220 lbs. She developed type II diabetes two years ago and was taking metformin. She was under supervision by a nurse at the diabetes clinic. She had tried most of the known diet programs but all failed. She began to take N-acetylcysteine 1200 mg in the mornings and evenings, usually before food craving. She took N-acetylcysteine 1200 mg in the afternoons, mostly after food craving. She lost 6 lbs two weeks after taking N-acetylcysteine. The next 2-weeks she did not lose or gain weight. She no longer had craving in between meals. She said that craving usually subsided within 15 minutes of taking N-acetylcysteine.

In light of the decreased food cravings and compulsive overeating as described above, one embodiment of the invention provides a method for decreasing food cravings and compulsive overeating in a patient comprising administering to the patient an effective amount of N-acetylcysteine or a pharmaceutically acceptable salt thereof.

### EXAMPLE 7

### Effect of N-acetylcysteine on Sexual Cravings and Hypersexual Behaviors

N-acetylcysteine reduced sexual cravings and hypersexual behaviors as exemplified in the case below.
A 36-year-old physically healthy man was seen for sexual addiction treatment. He had sexual arousal even at age five to six years old. He started to masturbate several years later. Pretty soon masturbation became compulsive daily and multiple times a day. By then he had more access to pornography and internet. He would spend hours and hours on it. His personal life was devastated. He had been in 12 step program called “Sex Addicts Anonymous” for the past 10 years in one of the largest cities in the US. Problem would repeatedly haunt him. Although he was given various antidepressants by doctors his condition had never stopped. He was treated with naltrexone and topiramate. These drugs treated his symptoms only partially. When N-acetylcysteine 2400-3600 mg/day was added, his symptoms were ameliorated dramatically. His hypersexual cravings and symptoms were gone. Currently, he is happily married and doing well at work. He is no longer taking naltrexone or topiramate. He is taking N-acetylcysteine only on an as needed basis. We have found same principles in voyeurism, exhibitionism and other sexual deviations. These patients often end up in jail yet they can't help themselves because of uncontrollable cravings. We found that N-acetylcysteine works dramatically in this group of patients.

In light of the decreased sexual cravings and hypersexual behaviors as described above, one embodiment of the invention provides a method for decreasing sexual cravings and hypersexual behaviors in a patient comprising administering to the patient an effective amount of N-acetylcysteine or a pharmaceutically acceptable salt thereof.

### EXAMPLE 8

### Effect of N-acetylcysteine on Shopping Cravings and Compulsive Shopping

N-acetylcysteine reduced shopping cravings and compulsive shopping as exemplified in the case below.
A 59-year-old well-educated woman was seen for compulsive shoplifting and shopping. She has had compulsive shopping behaviors for the past 20 years. She has been under severe financial stress because of her spending habits and unemployment. She had other addictive behaviors such as drinking, hoarding and overeating. Earlier, she was married, had stable job but lost everything. Various medicines such as antipsychotics and antidepressants were ineffective. On the third visit she was given N-acetylcysteine and was advised to take 2400-3600 mg/day. Her shopping cravings and behaviors stopped dramatically within two weeks.

In light of the decreased shopping cravings and compulsive shopping as described above, one embodiment of the invention provides a method for decreasing shopping cravings and compulsive shopping in a patient comprising administering to the patient an effective amount of N-acetylcysteine or a pharmaceutically acceptable salt thereof.

### REFERENCES

1. Ronis M J, Butura A, Sampey B P, et al. Effects of N-acetylcysteine on ethanol-induced hepatotoxicity in rats fed via total enteral nutrition. Free Radic Biol Med 2005; 39(5):619-30.
2. Pascale R, Daino L, Garcea R, et al. Inhibition by ethanol of rat liver plasma membrane (Na+,K+)ATPase: protective effect of S-adenosyl-L-methionine, L-methionine, and N-acetylcysteine. Toxicol Appl Pharmacol 1989; 97(2):216-29.
3. Aydin S, Ozaras R, Uzun H, et al. N-acetylcysteine reduced the effect of ethanol on antioxidant system in rat plasma and brain tissue. Tohoku J Exp Med 2002; 198(2):71-7.
4. Atkins K B, Lodhi I J, Hurley L L, Hinshaw D B. N-acetylcysteine and endothelial cell injury by sulfur mustard. J Appl Toxicol 2000; 20 Suppl 1:S125-8.
5. Menor C, Fernandez-Moreno M D, Fueyo J A, et al. Azathioprine acts upon rat hepatocyte mitochondria and stress-activated protein kinases leading to necrosis: protective role of N-acetyl-L-cysteine. J Pharmacol Exp Ther 2004; 311(2):668-76.
6. Raza M, Ahmad M, Gado A, Al-Shabanah O A. A comparison of hepatoprotective activities of aminoguanidine and N-acetylcysteine in rat against the toxic damage induced by azathioprine. Comp Biochem Physiol C Toxicol Pharmacol 2003; 134(4):451-6.
7. Labib R, Abdel-Rahman M S, Turkall R. N-acetylcysteine pretreatment decreases cocaine and endotoxin-induced hepatotoxicity. J Toxicol Environ Health A 2003; 66(3):223-39.
8. Sprague C L, Elfarra A A. Protection of rats against 3-butene-1,2-diol-induced hepatotoxicity and hypoglycemia by N-acetyl-l-cysteine. Toxicol Appl Pharmacol 2005; 207(3):266-74.
9. Peterson T C, Brown I R. Cysteamine in combination with N-acetylcysteine prevents acetaminophen-induced hepatotoxicity. Can J Physiol Pharmacol 1992; 70(1):20-8.
10. Kalivas P W, Volkow N D. The neural basis of addiction: a pathology of motivation and choice. Am J Psychiatry 2005; 162(8):1403-13.
11. Gass J T, Olive M F. Glutamatergic substrates of drug addiction and alcoholism. 2007.
12. Zhou W, Kalivas P W. N-Acetylcysteine Reduces Extinction Responding and Induces Enduring Reductions in Cue- and Heroin-Induced Drug-Seeking. 2007.
13. Baker D A, McFarland K, Lake R W, et al. Neuroadaptations in cystine-glutamate exchange underlie cocaine relapse. Nat Neurosci 2003; 6(7):743-9.
14. Baker D A, McFarland K, Lake R W, Shen H, Toda S, Kalivas P W. N-acetyl cysteine-induced blockade of cocaine-induced reinstatement. Ann N.Y. Acad Sci 2003; 1003:349-51.
15. Mardikian P N, LaRowe S D, Hedden S, Kalivas P W, Malcolm R J. An open-label trial of N-acetylcysteine for the treatment of cocaine dependence: a pilot study. Prog Neuropsychopharmacol Biol Psychiatry 2007; 31(2):389-94.
16. LaRowe S D, Mardikian P, Malcolm R, et al. Safety and tolerability of N-acetylcysteine in cocaine-dependent individuals. Am J Addict 2006; 15(1):105-10.
17. LaRowe S D, Myrick H, Hedden S, et al. Is cocaine desire reduced by N-acetylcysteine? The American journal of psychiatry 2007; 164(7):1115-7.
18. Grant J E, Kim S W, Odlaug B L. N-acetyl cysteine, a glutamate-modulating agent, in the treatment of pathological gambling: a pilot study. Biol Psychiatry 2007; 62(6):652-7.
19. Seiva F R, Amauchi J F, Rocha K K, et al. Alcoholism and alcohol abstinence: N-acetylcysteine to improve energy expenditure, myocardial oxidative stress, and energy metabolism in alcoholic heart disease. Alcohol 2009; 43(8):649-56.
20. Ferreira Seiva F R, Amauchi J F, Ribeiro Rocha K K, et al. Effects of N-acetylcysteine on alcohol abstinence and alcohol-induced adverse effects in rats. Alcohol 2009; 43(2):127-35.

All publications, patents, and patent documents are incorporated by reference herein, as though individually incorporated by reference. The invention has been described with reference to various specific and preferred embodiments and techniques. However, it should be understood that many variations and modifications might be made while remaining within the spirit and scope of the invention.

What is claimed is:

1. A method for treating alcohol-related disorders in a patient comprising administering to a human an effective amount of N-acetylcysteine or a pharmaceutically acceptable salt thereof to the patient to treat one or more disorders, wherein the disorders comprise alcohol dependence, alcohol abuse, alcohol intoxication, alcohol withdrawal, alcohol-induced delirium, alcohol-induced persisting dementia, alcohol-induced persisting amnestic disorder, alcohol-induced psychotic disorder, alcohol-induced mood disorder, alcohol-induced anxiety disorder, alcohol-induced sexual dysfunction or alcohol-induced sleep disorder.

2. The method of claim 1 wherein N-acetylcysteine is administered orally, parenterally, by injection or infusion, by an extended-release formulation, by a transdermal patch, or by inhalation.

3. The method of claim 1 wherein the effective amount of N-acetylcysteine administered orally is about 600-8000 mg.

4. The method of claim 3 wherein the effective amount of N-acetylcysteine administered orally is about 1200-4800 mg.

5. The method of claim 4 wherein the effective amount of N-acetylcysteine administered orally is about 2400-3600 mg.

6. The method of claim 1 the administration of N-acetylcysteine further improves hepatic function, wherein the improved hepatic function is a decreased level of a liver enzyme comprising aspartate aminotransferase, alanine aminotransferase or γ-glutamyl transferase, wherein the hepatic function by a liver function test comprising an aspartate aminotransferase liver function test, an alanine aminotransferase liver function test or a γ-glutamyl transferase liver function test.

7. The method of claim 1 wherein the administration of N-acetylcysteine further decreases alcohol use.

8. A method for preventing alcohol-related disorders in a patient comprising administering an effective amount of N-acetylcysteine or a pharmaceutically acceptable salt thereof to the patient to prevent one or more disorders, wherein the disorders comprise alcohol dependence, alcohol abuse, alcohol intoxication, alcohol withdrawal, alcohol-induced delirium, alcohol-induced persisting dementia, alcohol-induced persisting amnestic disorder, alcohol-induced psychotic disorder, alcohol-induced mood disorder, alcohol-induced anxiety disorder, alcohol-induced sexual dysfunction or alcohol-induced sleep disorder.

9. The method of claim 8 wherein N-acetylcysteine is administered orally, parenterally, by injection or infusion, by an extended-release formulation, by a transdermal patch, or by inhalation.

10. The method of claim 8 wherein the effective amount of N-acetylcysteine administered orally is about 600-8000 mg.

11. The method of claim 10 wherein the effective amount of N-acetylcysteine administered orally is about 1200-4800 mg.

12. The method of claim 11 wherein the effective amount of N-acetylcysteine administered orally is about 2400-3600 mg.

13. The method of claim 8 wherein the administration of N-acetylcysteine further improves hepatic function, wherein the improved hepatic function is a decreased level of a liver enzyme comprising aspartate aminotransferase, alanine aminotransferase or γ-glutamyl transferase, wherein the hepatic function by a liver function test comprising an aspartate aminotransferase liver function test, an alanine aminotransferase liver function test or a γ-glutamyl transferase liver function test.

14. The method of claim 1 wherein the administration of N-acetylcysteine further decreases alcohol use.

15. A method for treating or preventing craving-related disorders in a patient comprising administering an effective amount of N-acetylcysteine to the patient to treat or prevent one or more craving disorders, wherein the craving disorders comprise food cravings and compulsive overeating, sexual cravings and hypersexual behaviors, or shopping cravings and compulsive shopping.

16. The method of claim 15 wherein N-acetylcysteine is administered orally, parenterally, by injection or infusion, by an extended-release formulation, by a transdermal patch, or by inhalation.

17. A method of treating a hospitalized patient diagnosed with alcohol-related disorders or alcohol-related cell injury comprising administering 600-8000 mg/day to the patient in an emergency department, detoxification unit or inpatient settings.

18. The method of claim 17 wherein the effective amount of N-acetylcysteine administered orally is about 1200-4800 mg.

19. The method of claim 18 wherein the effective amount of N-acetylcysteine administered orally is about 2400-3600 mg.

20. The method of claim 17 wherein N-acetylcysteine is administered parenterally to a patient in an intensive care unit alone or together with benzodiazepines, thiamine, and folic acid for treating alcohol-related disorder or alcohol-related cell injury.
